(19) United States
(12) Patent Application Publication
(10) Pub. No.: US 2019/0117317 A1
(43) Pub. Date: Apr. 25, 2019

(54) ORGAN MOTION COMPENSATION

(71) Applicants: CANON U.S.A., INC., Melville, NY (US); THE BRIGHAM AND WOMEN'S HOSPITAL INC., Boston, MA (US)

(72) Inventors: Momen ABAYAZID, Enschede (NL); Nobuhiko HATA, Newton, MA (US); Takahisa KATO, Brookline, MA (US); Brian NINNI, Brighton, MA (US); Junichi TOKUDA, Newton, MA (US); Stuart G. SILVERMAN, Brookline, MA (US); Zhimin LU, Chelmsford, MA (US)

(21) Appl. No.: 16/093,405

(22) PCT Filed: Apr. 11, 2017
(86) PCT No.: PCT/US17/27037

§ 371 Date: Oct. 12, 2018

Related U.S. Application Data
(60) Provisional application No. 62/321,495, filed on Apr. 12, 2016.
(60) Provisional application No. 62/372,541, filed on Aug. 9, 2016.

(51) Int. Cl. A61B 34/20; A61B 5/00; A61B 5/113; A61B 10/02; A61B 18/14; A61B 5/06

(52) U.S. Cl. CPC A61B 34/20; A61B 5/7267; A61B 5/113; A61B 2017/00699; A61B 18/1477; A61B 5/066; A61B 10/0233

(57) ABSTRACT

A method and system for motion tracking of a target organ are provided. A tracking needle is partially inserted into an organ. The needle has a sensor element for obtaining continuous needle orientation information which can be used to determine organ motion due to respiration during percutaneous needle insertion.

### CROSS-REFERENCE TO RELATED APPLICATION(S)

This application claims priority to co-pending U.S. Provisional Application Ser. No. 62/321,495 filed Apr. 12, 2016, and to co-pending U.S. Provisional Application Ser. No. 62/372,541 filed Aug. 9, 2016, the contents of each of which are incorporated herein by reference in their entirety.

### STATEMENT REGARDING FEDERALLY SPONSORED RESEARCH

The government may own rights in this invention pursuant to National Institute of Biomedical Imaging and Bioengineering of the National Institutes of Health under award number P41EB015898.

### FIELD OF THE DISCLOSURE

The present disclosure relates to medical devices. More particularly, the disclosure exemplifies an apparatus and method for evaluating the motion of an organ, such as during respiration.

### BACKGROUND INFORMATION

Percutaneous needle insertions into organs such as the liver are common minimally invasive surgical procedures. Needles are often used for diagnostic and therapeutic applications such as biopsy and ablation, respectively. Clinical imaging techniques such as ultrasound, magnetic resonance images, computed tomography (CT) scans and cone beam CT's taken by fluoroscopes can be used during needle insertion procedures to obtain the needle and target positions. The success of the procedure is dependent on the accuracy of needle placement. Inaccurate needle placement can cause misdiagnoses and insufficient treatment in case of biopsy and ablation, respectively. Targeting inaccuracy can be caused by patient motion during the procedure and physiological processes such as fluid flow and respiration.

Respiratory motion is considered to be the main cause of inaccurate needle placement, especially in liver biopsy. Since the liver is located directly beneath the diaphragm, it is strongly influenced by the respiratory motion. The liver is pushed in inferior direction during inhalation as the diaphragm contracts, and it moves in superior direction during exhalation. The liver shows large variations in size and shape between subjects and the branching topology of the blood vessels and the biliary ducts can also show a variety of anomalies. Therefore, it is a challenging task to establish a standard anatomical atlas of the liver that is applicable to all subjects. For tumors in the liver, Kitamura et al. (IJRO Biology*Physics 56(1), 221-228 (2003)) showed that the extent of motion depends to a certain degree on their position in the liver, cirrhosis (late stage of scarring), and the surgical history. However, these factors are not sufficient to predict the motion of a target in the liver. Patient-specific assessment of the respiratory motion is therefore highly desired.

Breath-hold is the most common method to compensate for respiratory motion. The main disadvantage of this method is that in some cases the patient cannot hold his breath for sufficient time. Additionally, this technique is not suitable when automation is incorporated into the system where the physician's knowledge of the phase of the breath is not integrated into the workflow. Other motion compensation techniques have been developed to track respiratory motion. These techniques include a piezo-electric belt positioned around a person's chest or stomach to obtain a surrofate. Belt movement stretching the belt and creates a measurable electric charge which is used as a surrogate signal. Another technique is a breathing bellows that positions a small accordion tube around a patient's chest and is connected to a pressure sensor. Other techniques are based on gating and marker tracking and radiation control.

However, many of these techniques are not suitable for use with imaging—either due to the presence of metal or the device size is not compatible with the instrument.

Further, the use of guidance devices such as navigation software, needle guide mesh, and stereotactic needle guides (manual or robotic) is also important since guidance devices are increasingly being used to assist the accuracy of needle placement. However, these devices often do not have the ability to deal with motion of the target organ to which the intervention is applied on. This becomes particularly relevant when the patient either cannot or is uncooperative in breath holding. In these instances, the inability of the guidance device and methods to align the intervention tool to the pre-planned tool approach path to update the desirable approach path based on the location of the moving organs is particularly problematic.

Additionally, these devices and methods make various presumptions about respiration and the relative movement of the patient. For example, while the actual location of a target position such as a tumor is desired, these methods instead measure such things as the topical motion of skin under a belt, the change in muscle movement near the surface of the skin. Thus there is a reduced accuracy with which these devices and methods provide for the location of an organ during respirations compared to a more direct measurement of organ motion.

Thus, there is need for a new technique to overcome the problems mentioned above and to provide accurate and/or precise information for evaluating the motion of an organ.

### SUMMARY OF EXEMPLARY EMBODIMENTS

According to at least one embodiment of the invention, there is provided a motion tracking method comprising: inserting a tracking needle partially into an organ, wherein a sensor element is attached to the tracking needle; obtaining at least one image of the tracking needle and at least part of the organ; obtaining information of at least one target position from an image; obtaining continuous needle orientation information (as surrogate real-time signals) from the sensor element; correlating the surrogate signals (needle orientation) with target position to obtain a correspondence model; and determining the instantaneous location of the target position based on the correspondence model. The method may comprise obtaining a plurality of images within one breath cycle. A machine learning algorithm may be used for the correlation where the training set includes the plurality of images and continuous needle orientation information.

Other embodiments of the invention include a motion tracking system, comprising: a tracking needle; a sensor element attached to the tracking needle; a data processing system and an output device comprising a display or a needle guidance device. The data processing system is configured to: obtain continuous needle orientation information from the sensor element; correlate the measured surrogate signals from the sensing element (needle orientation) with a target position to obtain a correspondence model; and determine the instantaneous locations based on the correspondence model.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

Further objects, features and advantages of the present disclosure will become apparent from the following detailed description when taken in conjunction with the accompanying figures showing illustrative embodiments of the present disclosure.

FIG. 1(A) is a diagram of an embodiment showing a tracking needle inserted into a patient. FIG. 1(B) provides a closer view of the tracking needle. FIG. 1(C) is a diagram of an embodiment showing a tracking needle and includes a second therapeutic needle.

FIG. 2 is a diagram of an embodiment showing a tracking needle and a needle guidance device.

FIG. 3 is a diagram of an inserted tracking needle shown respiration.

FIG. 4 is a diagram including the motion tracking system.

FIG. 5 is a diagram of an embodiment of the invention including a gyroscope.

FIG. 6 is a diagram of an embodiment of the invention including a gyroscope.

FIG. 7 is a computed tomography (CT) image of liver including the marked location of a particular point in the liver.

FIG. 8 is a diagram of the experimental setup for Example 3.

FIG. 9 is a diagram of the experimental setup having a pre-processing unit and a learning-based algorithm.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENTS

A patient-specific approach to measure the target motion during respiration is provided herein. A sensor element configured to provide orientation information is attached to the tracking needle to measure the needle deflection outside the patient's body. This surrogate signal is correlated with information on organ motion obtained from one or more images of the organ in which the organ is inserted. The correlation is done using an algorithm suitable to estimate the target motion during respiration that estimates motion of the target, and consequently provides accurate needle placement and thus improve the clinical outcome of percutaneous needle interventions. The algorithm can use an inertial measurement unit (IMU) to measure the orientation change of the tracking needle inserted (prior to the actual targeting insertions) in the moving liver to estimate the liver's motion during respiration. Furthermore, a machine learning algorithm can be the correspondence model that correlates the surrogate signal (IMU data) to the actual target motion during respiration.

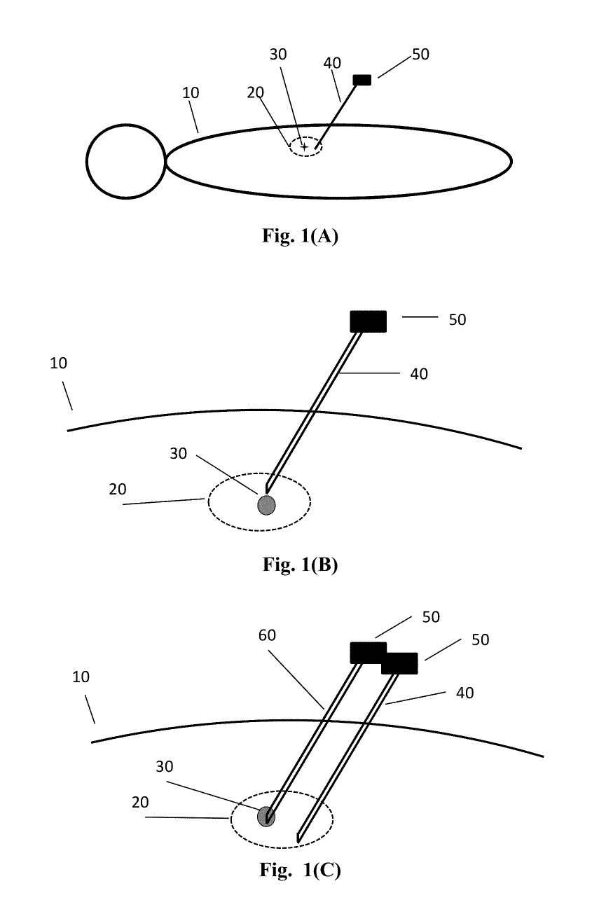

Thus, as provided in FIGS. 1(A) and 1(B), a patient 10 has an internal organ 20 that contains a lesion 30. The patient may be imaged to determine where the lesion 30 is located within the organ 20 and the location of a target position in the lesion 30. A tracking needle 40 having an attached sensor element 50 is inserted into the internal organ 20. There may also be a second needle 60 that may or may not also have a sensor element 50. This second needle 60 is used for a therapeutic purpose such as for a biopsy or ablation. Because of the surrogate signal gained from the sensor element located on the first tracking needle 40, the second needle 60 can more accurately be placed into the target position 30.

After insertion of the tracking needle 40, one or more additional therapeutic needles 60 (see FIG. 1(C)) may similarly be inserted at the target position 30 or alternatively into other target positions within the lesion. These additional needles optionally also include a sensor 50 that can provide additional information about the needle location, orientation, displacement, etc. However, the inclusion of a sensor 50 on the therapeutic needle is not required.

The tracking needle 40 as used herein is a slender instrument having a tip adapted for puncture. It may be, for example, a biopsy needle, a needle without a hollow core, or a cannula sleeve. A needle with a shape or size different from those described herein may be used. In some embodiments, the tracking needle must be somewhat rigid such that there is not significant deformation in the needle when partially inserted into an organ. For example, an 18, 20, or 22 gauge needle may be used. However, in other embodiments, a more flexible (e.g., thinner and less invasive) needle may be used when a deflection sensor is attached to the needle and defection surrogate signal is used to compensate for needle deflection.

The tracking needle is partially inserted into an organ. Thus, at least a portion of the needle is within the organ and a portion of the needle is outside the organ. The portion of the needle containing the sensor element remains outside of the patient during the methods as disclosed herein. In some embodiments, it is preferred to insert the needle substantially into—but not completely through the organ. The needle may be inserted into the patient directly above the organ or the needle may be inserted at an angle.

The sensor element 50 is attached to the proximal end of the tracking needle 40 provides position information, where position information includes information at least about rotation around x₁ and y₁ axes, where the tracking needle insertion axes is defined as z₁. Position information may also include information about rotation around the z₁ axis. Additionally, the sensor element may provide additional information such as translational information along the x₂, y₂, and/or z₂ axes, where z₂ is the axis tangential to the patient at the point of needle insertion. For embodiments providing liver motion information, information from the x₂ and z₂ directions are preferred, where x₂ is the vertical or longitudinal axis. The sensor element may be attached to the tracking needle by, for example: glue, tape, adhesives, magnetism, vacuuming, locking mechanisms, or via the addition of an adaptor or holder piece or multiple holder pieces. The sensor element may be embedded within needle. In some embodiments, the sensor element may be attached to the tracking needle after the needle is inserted into the organ.

The sensor or sensors may include, for example, an electromagnetic sensor, an optical sensor, an acoustic sensor, an electromagnetic (EM) sensor (e.g., a local EM sensor), an ultrasonic sensor, a PIR motion sensor, a displacement sensor, an inertial sensor, an accelerometer, a magnetometer, or a gyroscopic sensor. Multiple sensor elements may be provided instead of a single sensor element. In some embodiments, the sensor element on the needle is an emitter and a transceiver is located, for example, on the patient or on a needle guidance device. In some embodiments, the sensor element on the needle is a transceiver and an emitter is located, for example, on the patient or on a needle guidance device. In yet other embodiments, the sensor contains only a single sensor element located on the needle. When two or more needles are used and each needle has a unique sensor, those sensors may both be emitters and a single transceiver is located on the patient or on a needle guidance device.

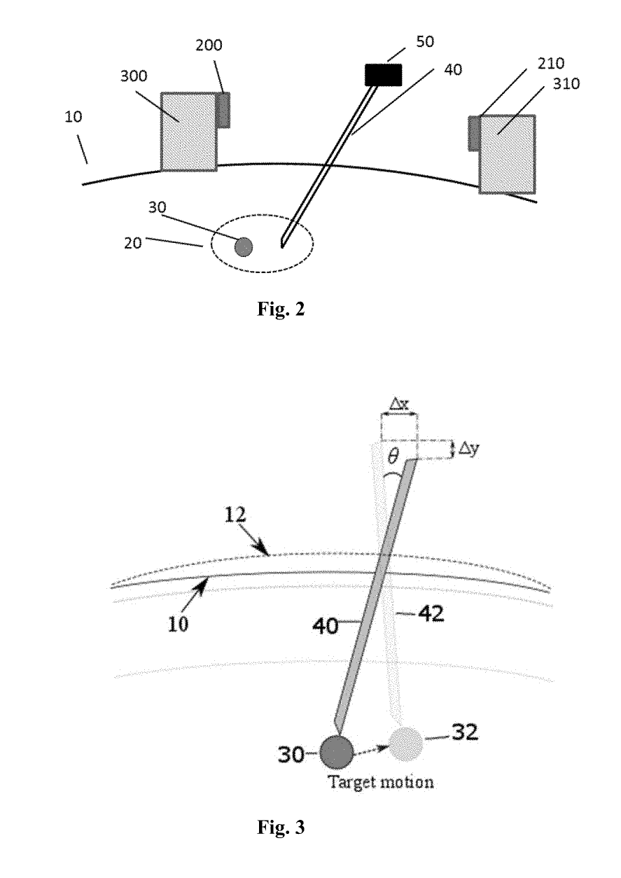

In some embodiments, a sensor element is on the tracking needle and a sensor element is on a needle guidance device that has a ring (or dual ring) shape such as described in U.S. Pat. No. 9,222,996. As shown in FIG. 2, the apparatus shown in FIG. 1 further comprises a needle guidance device having a ring shape, where the two sides of the rings 300 and 310 shown in a cross-sectional view, surround the tracking needle. Attached to the rings, a sensor element 200 and 210 is shown. This sensor element may have a ring shape or a partial ring shape and extend around the ring. This sensor element may be a smaller sensor located in only a single location or it may comprise multiple sensor element parts around the ring. This embodiment has particular advantages since the needle guidance device is attached to the patient. Thus, the sensor element(s) 200, 201 are attached to the frame of reference. Any gross movements of the patient's body will effect he same gross movements of the sensor element(s) (200, 210). Additionally, since the needle guidance device will have been registered with the patient, less additional registration needs to be performed. In one example, an optical sensor is located on the ring.

In some embodiments, there is an additional sensor element, where the additional sensor element is used to detect any bending motion of the needle. This additional sensor element may also be attached to the tracking needle. Particularly for thinner needles, the needle may have a tendency to bend when inserted into a patient such that the tip of the needle that is located in an internal organ is not co-linear with the portion of the needle that remains outside of the patient. The additional sensor element can be used to determine whether this bending motion occurs and to compensate for this motion. The addition of this additional sensor element allows for the use of thinner needles and thus provides a less evasive procedure for the patient.

In some embodiments, the sensor element, or a second sensor element located on the tracking needle includes linear acceleration and/or rotational velocity information. This information can be used in addition to the orientation information when correlating the needle orientation information to obtain the instantaneous location of the target position.

In some embodiments, additional sensors or apparatus providing additional information may also be included in the method and systems as provided herein. In some embodiments, the additional sensor element is an ultrasonic sensor. This additional sensor could be used for tumor location and visualization.

In some embodiments, the additional sensor element is an electromyographic sensor (EMG). The addition of EMG information from the respiratory muscles can provide information about muscle movement, such as coughing.

### Organ Motion

The sensor element 50 attached to the tracking needle 40 can be used to determine organ motion. First, at least one image is obtained of the tracking needle in the organ. This image may be used by a clinician to determine the location of a target position, where the target position may be, for example, the point from which a biopsy will be taken or the point where ablation is needed. Alternatively, the location of a target point may be obtained from a prior image or via other means.

As shown in FIG. 3, the tracking needle 40 that is inserted into an organ near a target position 30 will move during inhalation and exhalation. This is demonstrated in FIG. 3 by the position of the patient's skin during exhalation 10 where the position of the needle 40 is shown. During inhalation, the patient's skin 12 is in a different location as is the target position 32. The position of the needle tip effects the anterior position of the needle which moves from a first location (needle 40) to a second location (needle 42) which can be described as an angle θ and also Δx and Δy.

The at least one image may be, for example, a CT or MRI image. In some embodiments, a plurality of images is obtained. For example, at least 5, or at least 8, or at least 10 images are obtained within a 5-second or a 10-second or a 30-second time period. The plurality of images allows for mapping over a breath cycle and form an image map. In some embodiments, the plurality of images is obtained within one breath cycle to form the image map. In other embodiments, images over several breath cycles may be used to form the image map for the motion during a breath cycle.

Next, orientation information from the sensor element is obtained, where the position motion may be obtained continuously to measure the position as it varies based on the respiration of the patient. The continuous needle orientation information represents a surrogate signal that may be obtained in real time or it may be done based on the breath cycle of the patient. For example, the orientation information may be obtained during respiratory pose, wherein respiratory pose may be determined from observation, prior imaging data, or other means. As used herein, “real time,” as used in the context of obtaining orientation information means that the information is obtained at least once every 3 seconds, at least once every 2 seconds, at least once every second, approximately once every 0.5 seconds, or less.

The needle orientation information is correlated with the with target position to obtain a correspondence model, where the correspondence model is used to determine the instantaneous location target position. This location will change with the respiration of the patient.

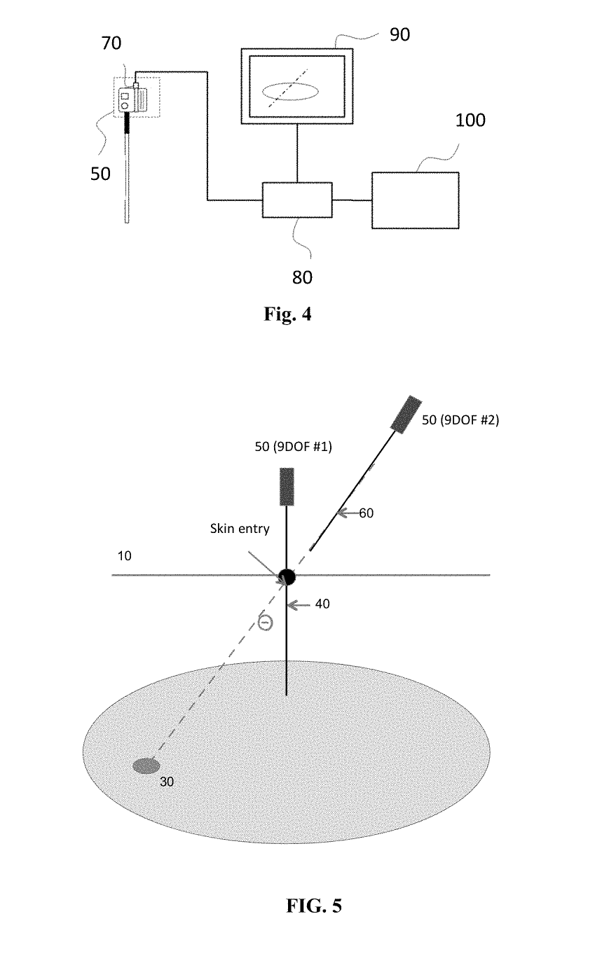

FIG. 4 shows an exemplary motion tracking system where the sensor element 50 is electrically connected to console 80 via signal interface 70. This connection also supplies a power for the sensor element 50. The console 80 may include a data processing system that can perform the correlation as described herein. Console 80 is further connected to an indicator 90 and image server 100. Console 80 receives medical images, for example CT or MRI images, from image server 100 that is, for example, a DICOM server connected to the motion tracking system. The medical images may be sent to indicator 90 with annotated information to help the physician to view, plan, or alter the medical procedure by console 80.

Also, console 80 and/or image server 100 can be adopted to interact with the physician to define a target position and/or a target trajectory with the medical images. For example, during planning, a physician can define the target position and/or target trajectory after viewing one or more medial image(s) on the image server 100. Several target positions and/or target trajectories can be defined for procedures that require more than one deployment (e.g., multiple needle placements). This information can be sent to the motion tracking system and the system can then aid the physician with determining the instantaneous location of the organ and the target position in that organ.

The system may also include a data storage unit, which may be included in the console or may be separate. This data storage unit stores the target position and/or the continuous needle orientation as well as the target trajectory for a needle to reach the target position. The orientation and/or trajectory information stored by the data storage unit can be used, for example, to evaluate similarity or discrepancy among the orientations at different time. By storing the target position, the motion tracking system can evaluate discrepancy between the target position and the continuous needle orientation. With this discrepancy, the physician or needle guidance device can know when it is appropriate to insert a second needle because the discrepancy due to respiration is minimized.

### Correlation

The data processing system is used to correlate the needle orientation information with the target position. This may be done, for example, by interpolation—by mapping the data against the respiratory phase. Alternatively, the correlation may be done using machine learning.

A learning-based approach may be used to determine organ motion and consequently the target motion during respiration. The needle orientations are initially measured while measuring the target motion. The recorded data is used for training in the learning-based algorithm to create a correspondence model that correlate the surrogate signal obtained from the sensor to the target position during respiration. A multi-variant regression method is then used to estimate the target motion using by using only the needle orientation as an input.

In some embodiments, training data including the target position (obtained from EM-sensor) and the needle orientation (from the orientation attached to the needle) are recorded for a period of at least 20 seconds (about 4 respiratory cycles). In some embodiments, the recording is for a period of at least 1, 2, 3, 4, 5, 6, 7. 8. 10, 15, 20, 25, or 30 seconds; or for 1, 2, 3, 4, 5, 6, 7, 8, 9, or 10 complete respiratory cycles). The recorded data can be synchronized and used for supervised training of the machine-learning algorithm.

In some embodiments, the (multi-label and multi-target) learning-based algorithm can be evaluated by splitting the data into training and testing points. The needle orientation data is then used as a numeric input to the trained system to estimate the 3D target position (numeric output). The multi-label learning-based classification software can be used to estimate the target position in 3D-space.

The learning-based algorithm can be evaluated by cross-validation of the training data points. The needle orientation data is then used as a numeric input to the trained system to estimate the target position (numeric output).

Thus, a patient-specific model is provided that uses an external orientation sensor to provide target motion during insertion. This external orientation sensor measures motion that is directly related to the organ motion due to respiration instead of, for example, motion of the patient's skin due to respirations and thus can provide the instantaneous location of a target position.

Thus, the methods and apparatus as provided herein provide the instantaneous location of a target position. This provides a good estimation of actual organ location since it is based on the actual location of the organ during respiration.

### Position Location

There are a number of applications where the instantaneous location of the target position (e.g., the estimated location) can be used. The instantaneous location of a target position during respiratory motion can be displayed to the clinician in real time. This can be performed virtually, for example, by placing a representation of the target inside a 3D model of the patient and having it constantly update to the estimated location. The 3D model of the patient can be created from the medical images of the patient, and can be hollow (only visualizing the skin surface) or show all of the anatomy. (Showing all of the anatomy might be confusing, however, since only the estimated target will be moving).

The instantaneous location of a target position can be used to indicate to the clinician whether or not the target is within a certain region of the anatomy at any given time. Inside the medical software, the clinician can specify a predefined volume inside of the anatomy (or a standard relative volume may be provided). This predefined volume may be set such that, given a predefined needle trajectory, if the instantaneous location of the target is within the predefined volume, the needle following the predefined needle trajectory will come sufficiently close to the target position. Thus, in some embodiments, whenever the instantaneous location of a target position is within this predefined volume, the clinician will be notified with some status indicator (for example, an audible or visual indicator). Whenever the instantaneous location of a target position leaves this volume, the clinician may again be notified. This will assist the clinician to select the optimal moment to insert the needle and place it accurately (e.g., during respiratory pause).

As one example, the motion estimation algorithm can determine the period where the target is along the needle trajectory. This information can then be used to give a green light to the clinician to insert the needle and during the period where the target moves away from the needle path due to respiration. The navigation system can also give the clinician a red light in order not to insert the needle until the organ moves. The status indicator (green and red lights) are exemplary located on a needle guidance device.

In some embodiments, the instantaneous location can be fed into a display in which an image having the target location is shown with the target location moving in real time. Thus, the physician can view the estimated location of the target location as it moves due to respiration.

Similarly, the volume location can be adjusted in real time. The location of this volume can be determined based on the position and orientation of another tracked device. For example, this can assist the clinician while inserting another needle into the target. If this new needle is similarly tracked, the software can extrapolate the trajectory of the needle and notify the clinician, in real time, whether or not the instantaneous location of a target position is within the needle trajectory's line of sight.

The instantaneous location of a target position can be, for example, fed into to a robotic device. The robotic device can constantly re-align its end-effector with the target position in real time during respiration. If the robotic device is only a guide for the clinician, it can indicate to the clinician, in real-time, whether or not the estimated target location is within its line of sight (or within a certain volume, which can be specific in the same manner as above). Alternatively, if the robotic device is an applicator, it will only ‘apply’ (insert the needle) at the instant where the target is aligned with the needle planned trajectory.

In addition to target position location, other positions may also be tracked using the methods and systems as provided herein.

The proposed approach can be used to model the motion of critical structures other than the target such as obstacles that need to be avoided by the needle during insertion. These obstacles can be sensitive tissue such as vessels and glands or a wall/membrane of a sensitive of organ that can be located along the needle trajectory at a certain instant during respiratory motion of organs. The critical structure information including its position and respiratory motion can be added to the machine learning algorithm as numeric inputs for training. This will create new correspondence model for each critical structure. The correspondence models can estimate the critical structure motion using the input surrogate data in real-time.

In some embodiments, additional sensors or apparatus providing additional information may also be included in the method and systems as provided herein.

In some embodiments, additional sensors or apparatus providing additional information (e.g., an EMG sensor) may also be included in the method and systems as provided herein. For example, the addition of EMG information from the respiratory muscles can provide information about muscle movement, such as coughing. Data from this additional sensor can be added to the correspondence model. For example, the sensor data can be used by the machine learning algorithm either or both as part of the training set or data used to obtain a correspondence model. The addition of such information can be used, for example, to enhance safety in an automated system, where needle injection is halted during any coughing or other contraction event(s). The addition of such information can be used, for example, to provide an improved training set. Thus, the data collected during any coughing event can be excluded from the training set images since the inclusion of such images could contain significant movements in addition to respiratory movements and increase the error in the correlation.

### Applications

The methods and systems as described herein may be used, for example, in a medical procedure such as a biopsy, ablation, or an injection. In these and other procedures, the organ position information can be used in a number of ways, including re-planning the clinical procedure, aborting the clinical procedure, deciding to continue the clinical procedure unchanged, and estimating organ movement.

Applications thus include the estimation of the target motion in liver induced by respiration during percutaneous needle intervention. The methods and systems as described herein use the motion of a reference needle as a surrogate signal and machine learning as a correspondence model to model estimate the target respiratory motion in liver. The motion of the reference needle can be measured using an IMU sensor attached to the needle hub.

Another application includes the recordation of organ movement over a population to map how the organ moves under certain conditions. Some conditions may related to certain procedures, like cryoablation, or other aspects of the procedure, like the patient lying position and if the patient is under general anesthesia or not. This map can be used to assist clinicians in preoperative planning.

### Systems

The embodiment(s) of the present invention also includes systems containing the motion tracking apparatus and a data processing system, such as a workstation. The data processing system includes conversion software and may also include visualization software, data logging software, and/or research software.

The data processing system may be, for example, connected to the sensor element directly or through a tracking software. The connection between the conversion software and tracker/tracking software may be created through, for example, universal serial bus (USB), serial, Ethernet, Bluetooth, wireless, or TCP/IP.

The invention further includes an apparatus and/or process to allow for the clinical staff to perform intra-procedural planning updates based upon how an organ has moved. For example, after a first needle has been inserted during an ablation, the planned placement of the subsequent needles is changed to account for organ motion.

Embodiment(s) of the present invention comprising one or more of the data processing system, the console 80, the image server 100, and optionally the indicator 90 can also be realized by one or more computer units CU that reads out and executes computer executable instructions (e.g., one or more programs) recorded on a transitory or non-transitory storage medium to perform the functions of one or more of the above-described embodiment(s) and/or that includes one or more circuits for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). In one embodiment console 80 is one computer unit and indicator 90 is a display unit connected to the console 80 via a high definition multimedia interface (HDMI), and the image server 100 is another computer unit connected to the console 80 connected via an Ethernet cable or the wireless access point.

The details of an exemplary computer unit CU are described. A Computer system includes at least one central processing unit (CPU), Storage/RAM (random access memory), I/O (input/output) Interface and user interface. Also, Computer system may comprises one or more devices. For example, the one computer may include the CPU, Storage/RAM, I/O Interface and other computers may include one or more user interfaces. The CPU is configured to read and perform computer-executable instructions stored in the Storage/RAM. The computer-executable instructions may include those for the performance of the methods and/or calculations described herein. For example, CPU calculates the center of the dark ring. Or, CPU calculates various values according to the information from the position sensor and/or other sensors, from the image server, from the signal interface. And so on. Storage/RAM includes one or more computer readable and/or writable media, and may include, for example, a magnetic disc (e.g., a hard disk), an optical disc (e.g., a DVD, a Blu-ray), a magneto-optical disk, semiconductor memory (e.g., a non-volatile memory card, flash memory, a solid state drive, SRAM, DRAM), an EPROM, an EEPROM, etc. Storage/RAM may store computer-readable data and/or computer-executable instructions. Each of components in the computer system communicates with each other via a bus. For example, the image date from, for example, a CT or MRI image is stored or sent through the image server 15 and may be stored in the storage/RAM. The image may then be displayed on a monitor with or without additional information from the medical guidance device 11 or user input such as a target orientation or discrepancy from the target orientation.

The I/O interface provides communication interfaces to input and output devices, which, in addition to the circuit board, indicator 14, the signal interface 12, and the image server 15, may include a communication cable, a network (either wired or wireless), or other devices. The user interface may be coupled to a user interface unit such as one or more of a keyboard, a mouse, a touch screen, a light pen, a microphone and so on.

The CU receives orientation information, such as the rotation information, the transitional information, motion information as described above. The information is received from the sensor element 50 attached to the tracking needle, via the I/O such as wired or wireless communication interface hardware. The CU may receive the patient motion information from the sensor element 200 and 210. The CU may perform other process for receiving the orientation information, as described above.

The CPU in the CU controls Storage/RAM to store the received orientation information. The sensor element detects multiple pieces of the orientation information at different points in time, and the CPU controls the Storage/RAM to store the multiple pieces of the orientation information.

Time information which indicates when each of the data is obtained may be associated with each of the orientation information and is stored in the storage/RAM. In one embodiment The CU may receive orientation information and the time information from the sensor element 50 via I/O Interface.

The CU also receives the operation input, for defining the target information, for example the target position, from the mouse or keyboard via the user interface. In the process of receiving the target information. The CPU in the CU causes the display unit to display the image(s), for example, a CT or MRI image acquired at a certain time. The image(s) may be a three-dimensional image of a target organ and may be axial, coronal or sagittal images. When the clinician uses a mouse or a keyboard to designate one point in the displayed image and make a click or push the button of the keyboard, the CU receives the input and acquires the position in the image via the I/O Interface. The CPU may associate time information indicating the image has been obtained with the acquired position, and controls the Storage/RAM to store the acquired position in the image as the target position, and to store the associated time information.

In the process, the clinician inputs multiple pieces of the target information for each image acquired at different points in time, and the multiple pieces of the target information are stored in the Storage/RAM.

In one embodiment the clinician may input the target trajectory information by inputting the position and the direction information. The CPU may perform other process for organ motion determination, as described above.

The CPU in the CU correlates at least a part of the stored multiple pieces of the needle orientation information and at least a part of the stored multiple pieces of the target information. The process may be done by using a machine learning algorithm such as support vector machine or some model fitting algorithm, to acquire a correspondence model. The correspondence model is stored in the Storage/RAM.

The correspondence model may be a mathematical matrix which can be used to output the estimated target position when needle orientation information is given as an input data. In one embodiment, if the basic model is given, the CPU acquires the parameters for modifying the basic model from the machine learning process. The CPU may perform other process for the correlation as described above.

After the correspondence model is acquired, the CPU in the CU determines the instantaneous location of the target position by the correspondence model. The CPU may cause the display unit to display the determined instantaneous location and the current orientation of the needle detected by the sensor element 50. The CPU may cause the display unit to present other information, as described above.

### Definitions

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated.

### EXAMPLES

### Example 1

In one example, a set of cross sectional images will be taken and stored in the external computer for intervention planning. The target location and skin entry points, as well as the approach line connecting these two controlling points are marked and digitized in the computer. The physician will be instructed to align the intervention device to the planned approach line with the help of position tracking sensor element attached to the intervention device. Alternatively, the stereotactic frame will be placed near the skin entry point, and approach path is provided by aligning intervention tool holder to the desirable tool approach path. The stereotactic frame can be motorized.

In the first instance, an in-situ sensor element is placed at the needle tip for direct motion tracking of target organ. The receiver can be placed on the abdomen. The receiver can then be registered to the cross sectional imaging system. Thus, when the needle is half inserted to an organ, in-situ sensor element at the tip of the needle indicates the location of the moving organ.

After the patient is out-side of the bore of the imaging modality, and instructed to hold the breath, the first needle is inserted, sensor element also records the location of the organ. The flow, for the insertion of two needles can comprise:

Liver Location B and Needle orientation B are recorded.

Patient is moved back into MRI for imaging.

Liver Location C and Needle orientation C are recorded.

Target Location C₁ is recorded.

Patient is moved outside the MRI and breath holding is instructed.

Liver Location D and Needle orientation D are recorded.

The new second needle should be inserted at the position D+(C−C₁)

Similarly, the needle tail may be used. In this aspect, a marker is placed on the tail of the half-inserted needle. A sensor element that is a transmitter/receiver can be placed on body, imaged with the lesion, and registered to the cross sectional imaging device. Then, the organ location is estimated presuming that the needle is considered to swivel along the needle insertion point. Conversely, if the needle is not straight and bends as the organ swivels, combined sensing of needle tip, such as by EM and tail, such as by optical method or EM sensing, can simulate the bending mode. The sensor element on the tail can be, for example, active marker, or led emitting transmitter. The on-body sensor element can be a receiver. The sensor element locations can also be flipped (e.g., the needle contains a receiver sensor element and the on-body sensor element is a transmitter). Either the needle based tracking or tail based tracking can be used with the needle guidance device described in U.S. Pat. No. 9,222,996.

### Example 2

In this example, the sensor element attached to the tracking needle is a 9 degree of freedom (9DOF) sensor. In addition to the positional information, this sensor or sensors include an inertial measurement unit with a 3-axis gyroscope, a 3-axis accelerometer and a 3-axis magnetometer. The sensors, devices, and methods as described in U.S. Provisional application Ser. No. 62/268,378 are herein incorporated by reference in their entirety.

FIG. 5 show the 9 DOF sensor attached to the tracking needle and a second 9 DOF sensor attached to the insertion needle. In real time, continue to feedback relative angle and error from the planed path when the tracking needle is moving, or in any phase of breath holding, provide the relative angle and planed position of relative angle at the entry point 110.

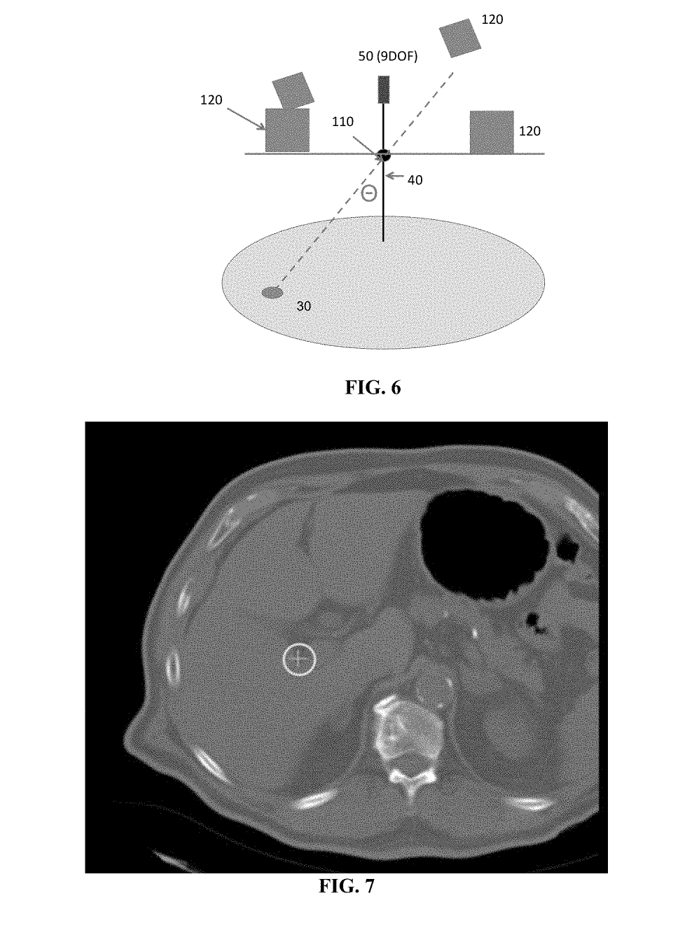

FIG. 6 shows a sensor in an insertion where a robot 120, which is also registered with the sensor/tracking needle with image registration. The solid squares indicate a sectional view of the two ring robot described in U.S. Pat. No. 9,222,996. The sensors sense the position information of both the tracking needle and an insertion needle. The sensor, which is shown as a 9 DOF sensor may include an inertial measurement unit with a 3-axis gyroscope, a 3-axis accelerometer and a 3-axis magnetometer and circuit board(s). The gyroscope, accelerometer and magnetometer may be incorporated onto a single chip with an integrated output, or they may be separate. This sensor also allows for reducing the drifting of the sensed orientation through long duration of usage for the operation

The tracking needle may be is invariant against skin entry point in translational motion.

There may be a small displacement assumption of organ against the tracking needle insertion, i.e. depth of position of target organ from skin surface. A viable assumption—particularly for the lever, is that the body of the organ is rigid, such that the positional relationship between tip of the tracking needle and target lesion (target position) is constant. Thus, we can assume small angle, i.e. theta˜tan theta˜sin theta. Therefore angle Lesion-Skin Entry-Tip Tracking Needle is constant in any position of moving organ.

### Example 3

Lesion Motion in Liver. In this example, clinical data was used to measure lesion motion in liver during respiration. Liver CT images of 64 scans obtained from four patients were used to measure the target motion in liver. The scans were obtained during liver biopsy and ablations cases. The patients did not move during the procedures and the main cause of motion in the liver was respiration. A structure that resembles the lesion was localized manually in each image frame of every CT scan (FIG. 7) using a free open-source medical image computing software, 3D Slicer. The localization method was examined and reviewed by board-certified physician in general surgery with five years of experience in percutaneous ablation therapies. The results show that the target maximum absolute displacement was 12.56 mm. The main component of this motion was a superior-inferior shift 5.5±3.84 mm. The liver additionally showed motion in anterior-posterior 3.77±2.07 mm and left-right direction (3.14±0.68 mm) These results were used as an input to the moving phantom designed to mimic the liver motion.

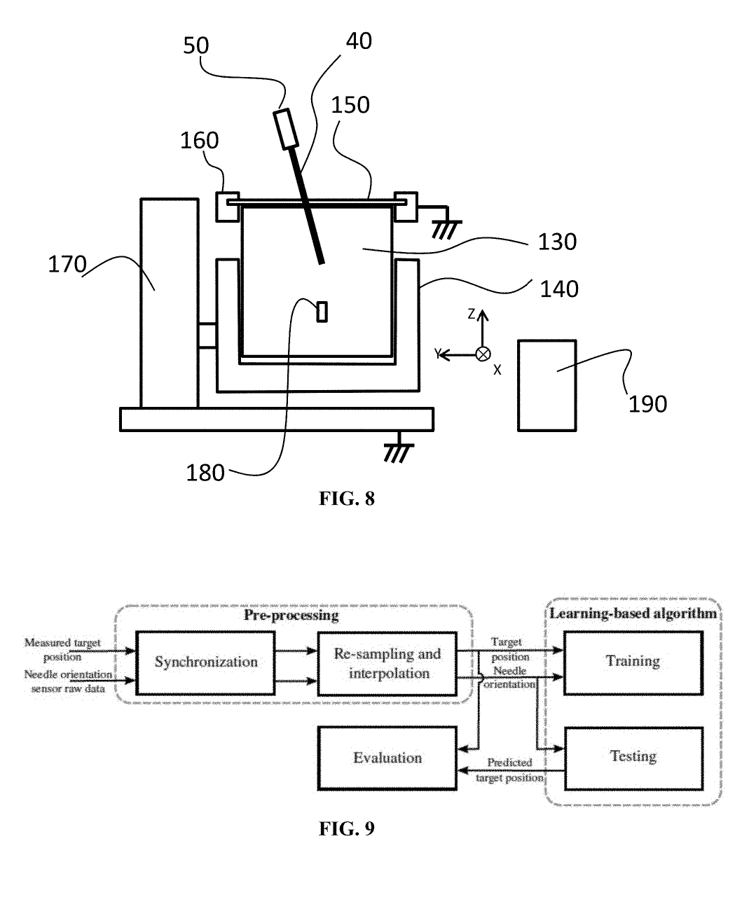

A gelatin-based phantom was used mimic the elasticity of human liver. The gelatin-to-water mixture of 15% (by weight) was used (Knox R gelatin, Kraft Foods Group, Inc., Ill., USA). The phantom was attached to two XZ motorized stages (type eTrack ET-100-11, Newmark Systems Group Inc. Calif., USA) actuated with stepper motors to simulate the respiratory target motion in liver. Both motors were controlled using a Newmark controller NSC-A2L Series. The setup for this experiment is shown in FIG. 8.

An abrasion-resistant natural latex rubber layer (McMaster-Carr, Ill., USA) of 0.5 mm thickness was used to mimic the skin. Aurora electromagnetic (EM) tracker (Northern Digital Inc., Waterloo, Canada) was used for measuring the needle position and orientation and also the target position with a frequency of 25 Hz. A tracking sensor was embedded into the tracking needle inserted in the gelatin phantom at different depths. The SDOF EM sensor was embedded in an 18 gauge needle to track its tip location and orientation. The 3D position, pitch and yaw angles were measured by the system. For this phantom study, another 6DOF EM sensor was embedded into the gelatin to measure the target motion. An orientation sensor was attached to the needle hub outside the patient's body. The orientation sensor (BNO055, Bosch Sensortec GmbH, Reutlingen, Germany) was composed of an advanced triaxial 16 bit gyroscope, a triaxial 14 bit accelerometer and a full performance geomagnetic sensor. The orientation sensor measured with frequency of 100 Hz. The actual target position was measured using the EM tracker a the target site and used as a gold standard. The EM and the orientation sensor measurements were synchronized and used as an input to the learning-based algorithm described in the following section.

Learning Algorithm. Training data including the target position (obtained from EM-sensor) and the needle orientation (from the orientation attached to the needle) are recorded for a period of 20 seconds (about 4 respiratory cycles). The measured data was synchronized and split where 66% of the collected data was use for training and 34% was used for testing the correspondence model (correlation). Random k-Labelsets method is used for classification (multi-variant regression. Disjoint labelset construction version RAkEL was used in the current study. See Tsoumakas G et al., (IEEE TKDE 23(7), 1079-1089 (2011) and Proc. ECML vol. 4701, pp. 406-417. Warsaw, Poland (September 2007)).

The (multi-label and multi-target) learning-based algorithm is evaluated by splitting the data into training and testing points. The needle orientation data is then used as a numeric input to the trained system to estimate the 3D target position (numeric output).

The machine learning algorithm used in this example is the open source MEKA software which is a multi-label version of WEKA that was developed at the Machine Learning Group at the University of Waikato in Hamilton, New Zealand. The multi-label learning-based classification software was used to estimate the target position in 3D-space.

Experiments. The most significant target motion due to respiration as understood for this experiment is in the superior-inferior direction. The respiratory cycle is composed of 1.5-2 s of inhalation, 1.5-2 s exhalation and then a pause of around 2 s. This motion pattern was simulated in the developed setup. The imposed target displacement was measured from the CT-data. Twenty seconds of target motion was recorded using an EM-tracker and needle orientation using both EM-tracker and orientation sensor. The needle was placed with varying proximity to the target. Two respiratory cycle durations were used for mimicking shallow breathing, with target motion magnitude of 12.56 mm.

Each experimental trial was performed at least 7 times. The Evaluation criteria included: position error, total processing time, the effect of the distance between the needle and the target, and also the effect of the respiratory duration on the estimation error.

The data obtained from the orientation sensor and the target motion (during ˜4 respiratory cycles) were used to train the learning-based approach to estimate the target motion during respiration. At respiratory cycle duration of 5 s, the median target estimation errors while using needle to target proximity range of 0-1 cm, 1-2 cm and 2-3 cm were 0 (o.55±0.85)mm, 0 (0.52±0.77)mm and 0 (0.74±0.99)mm (p=0.05), respectively, while at respiratory cycle duration of 6 s, the median target estimation errors were 0 (0.63±0.87)mm, 1 (0.74±0.79)mm and 0 (0.53±0.81)mm (p=0.003), respectively. Wilcoxon rank-sum, and Kruskal-Wallis tests were used to compare the errors.

The processing time for training and testing was 4-12 ms which is sufficient for real-time target motion estimation. The results show that respiratory cycle duration does not affect the error when the needle proximity to target is ≤1 cm (p=0.4) while at the proximity to target of >1 cm it significantly affects the estimation error (p<0.001 and 0.005). The needle proximity to target does significantly affect estimation error if the respiratory duration is 6 s. The proposed algorithm is expected to be generalized to compensate for target motion other organs.

### Example 4

The surrogate signal and the correspondence model used to estimate the target motion and also the liver MR data of human subject during respiratory motion is described. The motion data were used as input to the experimental platform used for the validation study.

Surrogate signal. To estimate the target motion, we used the external motion of the needle inserted into the moving organ as the surrogate signal. The surrogate signal is a measurable and accessible signal that can be used to estimate the actual target/organ motion. The surrogate signal is used in case, for example, the target location cannot be measured in real-time (within an acceptable delay) or if it cannot be located accurately due to respiratory motion artifacts in images and thus inaccurate target motion estimation. In this study, the external motion of the needle inserted into a moving organ was hypothesized to indicate its respiratory motion and consequently the target motion located in the liver, The concept of using the motion of the needle inserted into the moving organ as a surrogate signal to estimate the target motion in the liver was assessed using an IMU sensor. The sensor was attached to the needle hub outside the patient body. The IMU sensor (BNO055, Bosch Sensortec GmbH, Reutlingen, Germany) was composed of a triaxial 16bit gyroscope, triaxial 14 bit accelerometer and a geomagnetic sensor. The sensor measures the needle 3D orientation, linear acceleration and angular velocity around its pitch, roll and yaw axes with a frequency of 100 Hz. The IMU sensor was powered and controlled by a microcontroller board (Arduino MICRO, Arduino, Italy) via an Inter-Integrated Circuit module. Serial communication was used to connect the microcontroller board to the computer in order to record the measured data. The recoded data was used as an input to the learning-based algorithm to estimate the target motion in liver during respiration.

Correspondence model. The correspondence model attempts to model the relationship between the target motion in liver and the surrogate signal (IMU sensor data). This relationship was used to estimate the target motion based on the subsequent acquisition of the surrogate data. In this particular study, the correspondence model was trained using a machine learning algorithm. The actual target position represents the gold standard for training and also evaluation of the correspondence model. To train the correspondence model, the recorded surrogate signal and target motion data were synchronized and then used for supervised training of the machine learning algorithm (see FIG. 9). The machine learning method is based on Random k-Labelsets (RAkEL) method for classification (multi-vartiant regression). k is a parameter that specifies the size of the labelsets. An aspect of this method is to randomly break a large set of labels into a number of small-sized labelsets, and for each of labelsets train a multi-label classifier using the label powerset method. Disjoint labelset construction version RAkELd presented by G. Tsoumakas, et al., (IEEE Transactions on Knowledge and Data Engineering, vol. 23, no. 7, pp. 1079-1089, 2011) were used as it can handle multiple numeric inputs (surrogate data) and outputs (target position) within relatively short processing time. The (multi-label and multi-target) learning-based algorithm was evaluated by splitting the data into training and testing points. After training, the output correspondence model uses only the surrogate signal from the IMU sensor to estimate the three-dimensional position of the target at a certain moment during respiration. For training and testing the correspondence model, we used the open source MEKA software which is a multi-label version of WEKA that was developed at the Machine Learning Group at the University of Waikato in Hamilton, New Zealand. The multi-label learning-based classification software was used to generate the correspondence model and then estimate the target position in 3D-space.

FIG. 1 shows the needle orientation was measured for several respiratory cycles while measuring independently the target motion. As shown in FIG. 9, the measured needle orientation (sensor) and target position (electro-magnetic (EM) tracking) were pre-processed before being used for training the machine learning algorithm. The learning based algorithm was then tested to estimate the target motion using only the needle orientation as an input. The target position obtained from the EM sensor was used as the gold standard to evaluate the output of the learning-based algorithm.

Respiratory human MR data. Liver MR data of human subjects was used to determine the target motion profile in the liver during respiration. Eight human subjects were recruited and imaged following informed consent. MR liver sagittal scans obtained from the human subjects were used to measure the liver respiratory motion. The subjects did not move during the procedures and the main cause of motion in the liver was respiration. The subjects were scanner in 3T wide-bore MRI scanner (MAGNETOM Verio 3T, Siemens, Erlangen, Germany). The image slice thickness flip angle matrix size and field of view were 5 mm, 30°, 192×192 and 38×38 cm², respectively. The frequency of acquiring images was 1 Hz and the duration of each scan was 140.27±51.10 s. Per scan, 122±45.86 images were acquired. The MR images were acquired by a board-certified radiologist. In the liver MR images, the motion of three structures that resembles the lesion were tracked in each MR image frame. These structures were located manually in each image frame. The measured liver motion profile (that consists of the target displacement and velocity) was used as an input to the experimental platform designed to mimic the liver motion.

Validation study. The experimental platform (see FIG. 8) was designed to mimic the liver motion. The aim of performing the experiments is to validate the proposed surrogate signal and correspondence model at a variety of respiratory motion profiles and conditions such as the target depth, motion velocity, needle insertion angle and its proximity to the target. 1) Phantom: A gelatin-based phantom 130 was used mimic the elasticity of human liver. The gelatin-to-water mixture (1.6 L) of 15% (by weight) was used (KnoxR gelatin, Kraft Foods Group, Inc., Ill., USA). The phantom was placed in a container 140 and covered by an abrasion-resistant natural latex rubber layer 150 (McMaster-Carr, Ill., USA) of 0.5 mm thickness to mimic the skin. To simulate the respiratory motion, the skin layer and the upper part (2 cm) of the gelatin phantom were clamped in the x-y directions 160 but can move in the z 170 (up and down).

Experimental setup. The phantom 130 was attached to two motorized stages 170 (XZ) (type eTrack ET-100-11, Newmark Systems Group Inc. Calif., USA) actuated with stepper motors to simulate the respiratory target motion in liver. Both motors were controlled using a Newmark controller NSC-A2L Series (FIG. 8). The actual target position was measured using an electro-magnetic (EM) sensor (Northern Digital Inc., Waterloo, Canada) at the target site and used as a gold standard 180. Another 5 Degrees-of-Freedom (DoF) EM sensor 50 was also embedded into the reference needle 40 inserted in the phantom 130 to measure the distance between the needle and target, and also measure the needle insertion angle. The target location, the distance between the needle and target, and the needle insertion angle were displayed and calculated using a free open-source medical image computing software, 3D Slicer. The IMU sensor, was attached to the needle hub outside the phantom. The EM sensor data and the IMU sensor measurements were synchronized and used as an input to the learning-based algorithm (correspondence model). An electromagnetic field generator 190 is located outside the phantom.

Experimental protocol: Experiments were performed to determine the accuracy of the developed design at different insertion angles, target depths, target motion velocities and target proximity to the needle. The initial parameters are: 60° insertion angle, 8 cm target depth, 3.5 mm/s target motion velocity, 1-2 cm distance between the needle and target. The target motion range and the velocity were selected based on the results obtained from the MR-images. The range of target motion was randomized within the range obtained from the respiratory MR motion data. The experimental protocol is presented in Table I. Each experiment was repeated seven times. An extra experiment was performed in which both the range of target displacement and velocity are randomized within the range measured in the MR clinical data. This motion pattern was simulated in the experimental setup using the motorized stages. The duration of each experiment was 20 seconds of respiratory target motion. In each experiment, the target displacement was measured using the EM-tracker and needle motion was measured using the orientation sensor. Each experiment was performed seven times.

TABLE I
Experimental Protocol For Validation Of The Correspondence
Model While Varying The Motion Profiles And The Conditions

| | Insertion | | | Target | | | Motion | | | Proximity to | | |
|---|---|---|---|---|---|---|---|---|---|---|---|---|
| Experi- | angle (°) | | | depth (cm) | | | velocity (mm/s) | | | needle (cm) | | |
| ment | 40 | 60 | 90 | 4 | 8 | 16 | 2.5 | 3.5 | 4.5 | 0-1 | 1-2 | 2-3 |
| #1 | ✓ | ✓ | ✓ | | ✓ | | | ✓ | | | ✓ | |
| #2 | | ✓ | | ✓ | ✓ | ✓ | | ✓ | | | ✓ | |
| #3 | | ✓ | | | ✓ | | ✓ | ✓ | ✓ | | ✓ | |
| #4 | | ✓ | | | ✓ | | | ✓ | | ✓ | ✓ | ✓ |

Data analysis: In order to evaluate the developed design and algorithms, a number of parameters were selected for validation including the insertion angles, target depths, target motion velocities and target proximity to the needle. The accuracy of the estimated position of the target and the time required to process the data were the parameters used to validate the proposed concept of using the needle deflection (IMU data) to estimate the target position during respiratory motion.

The recorded data was synchronized and re-sampled using linear interpolation to obtain the same number of data points from both the target motion data (EM tracker) and needle motion (IMU sensor). The measured needle motion and target position at each instance during respiratory motion represent a single training point for the learning algorithm. The target position was then estimated by supervised training of data. 66% of the data points was used for training and 34% was used for testing the learning algorithm. The data points used for training and testing the learning algorithm were selected randomly in order to consider the variation in the respiratory motion profile in the collected data.

### Example 5

This example presents the results of the target tracking in the MR images used to generate the motion profile of the motorized phantom. The results of the correspondence model validation study are also presented.

Motion tracking in MR images. The tracked target motion in MR images showed that the motion in this example was mainly in the vertical and sagittal axis; 8.10±4.71 mm and 2.53±1.60 mm, respectively. The mean velocities of target motion were 3.54±1.04 mm/s and 1.11±0.33 mm/s in the vertical and sagittal axis, respectively.

Experimental target motion estimation. The results obtained from the learning algorithm are presented in Table II, where each experiment was repeated seven times and the estimation error is the absolute distance between the actual target and the target position and its estimated position at a certain moment during respiration.

TABLE II
Validation Of The Correspondence Model While Varying
The Motion Profiles And The Conditions Are Presented

| | Insertion | | | Target | | | Motion | | | Proximity to | | |
|---|---|---|---|---|---|---|---|---|---|---|---|---|
| Estimation | angle (°) | | | depth (cm) | | | velocity (mm/s) | | | needle (cm) | | |
| error (nm) | 40 | 60 | 90 | 4 | 8 | 16 | 2.5 | 3.5 | 4.5 | 0-1 | 1-2 | 2-3 |
| Mean | 1.08 | 1.04 | 0.86 | 1.08 | 1.04 | 1.08 | 1.10 | 1.04 | 0.90 | 1.29 | 1.04 | 1.21 |
| Standard | 0.48 | 0.46 | 0.53 | 0.43 | 0.46 | 0.46 | 0.49 | 0.46 | 0.44 | 0.37 | 0.46 | 0.41 |
| deviation | | | | | | | | | | | | |

Estimation error: The mean error of the absolute distance between the estimated position of the target was obtained from the learning-based algorithm and the actual position of the target measured using EM trackers embedded at the target site. The learning-based algorithm training time was in the range of 4 ms while the testing time was 1 ms.

Statistical analysis: Kruskal-Wallis test was performed to determine the statistical significance of the tested parameters. The results show that increasing of the insertion angle in the range between 40° and 90° increased the targeting error (p<0.005) while increasing the target depth in the range between 4 mm and 10 mm decreased the targeting error (p<0.005). It was observed that increasing the distance between the target and needle tip in the range between 0-1 cm and 2-3 cm increases the targeting error (p<0.005).

The experimental results show that the mean error of estimation of the target position ranges between 0.90-1.29 mm. The maximum time for training and testing the IMU data was 5 ms which is sufficient for real-time target motion estimation using the needle orientation (IMU) sensor. It was also observed that the estimation error of the target motion decreases as the target depth increases. The experimental conclusion is that when the needle is superficial it is more sensitive to factors other than the target motion such as the weight of sensors and cables attached to the needle since the needle is not deep and thus not well fixed to the moving organ. It was also observed that the error increases as the distance between the needle and target increases. Additionally, it was found that, varying the velocity of target motion did not show a significant change in the estimation error in the experiments described above.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

What is claimed is:

1. A motion tracking method comprising:
inserting a tracking needle partially into an organ, wherein a sensor element is attached to the tracking needle;
obtaining at least one image of the tracking needle and at least part of the organ;
obtaining information of at least one target position from an image;
obtaining continuous needle orientation information from the sensor element;
correlating needle orientation information with target position to obtain a correspondence model; and
determining the instantaneous location of the target position based on the correspondence model.

2. The method of claim 1, wherein obtaining at least one image comprises obtaining a plurality of images within one breath cycle.

3. The method of claim 1, wherein obtaining at least one image comprises obtaining at least 5 images over a time period of 3-30 seconds.

4. The method of claim 1, wherein obtaining information of at least one target position from an image comprise receiving location of a lesion from a clinician.

5. The method of claim 1, wherein correlating needle orientation information comprises using a machine learning algorithm.

6. The method of claim 5, wherein the machine learning algorithm uses a training set comprising target position information and continuous needle orientation information.

7. The method of claim 1, wherein continuous orientation information is obtained in real time.

8. The method of claim 1, wherein continuous orientation information is obtained during respiratory pose.

9. The method of claim 1, further comprising obtaining continuous translational information from the sensor element.

10. The method of claim 1, further comprising obtaining continuous rotational information from the sensor element.

11. The method of claim 1, further comprising: comparing the instantaneous location of the target position to a predefined volume and indicating relative position with a status indicator.

12. The method of claim 1, wherein a further sensor element is directly or indirectly attached to a patient.

13. The method of claim 1, further comprising: inserting a therapeutic needle at a location at least partially determined by the instantaneous location of the target position.

14. The method of claim 13, wherein a second sensor element is attached to the therapeutic needle.

15. The method of claim 1, wherein the image is an MRI or CT image.

16. A motion tracking system, comprising:
a tracking needle;
a sensor element attached to the tracking needle;
a data processing system configured to:
obtain continuous needle orientation information from the sensor element, correlate needle orientation information with a target position to obtain a correspondence model, and
determine the instantaneous locations based on the correspondence model; and
an output device comprising a display or a needle guidance device.

17. The motion tracking system of claim 16, further comprising a needle guidance device.

18. The motion tracking system of claim 17, wherein the needle guidance device comprises a sensor element.

19. The motion tracking system of claim 17, wherein the needle guidance device is configured to modify the orientation of a therapeutic needle based on the instantaneous location of the target position.

20. The motion tracking system of claim 17, wherein the needle guidance device comprises a therapeutic needle having a planned needle trajectory, and wherein the needle guidance device is configured to insert the therapeutic needle when the instantaneous locations obtained from the data processing system is aligned with the planned needle trajectory.
